(19) United States
(12) Patent Application Publication
(10) Pub. No.: US 2022/0408899 A1
(43) Pub. Date: Dec. 29, 2022

(54) FLUID DISCHARGER AND APPLICATOR DEVICE, METHOD FOR ASSEMBLING AND MANUFACTURING THE SAME AND ASSEMBLING SYSTEM

(71) Applicant: SELVA KUMAR, HOUSTON, TX (US)

(72) Inventor: SELVA KUMAR, HOUSTON, TX (US)

(73) Assignee: SELVA KUMAR, HOUSTON, TX (US)

(21) Appl. No.: 17/780,455

(22) PCT Filed: Jan. 14, 2021
(86) PCT No.: PCT/US21/13373

§ 371 Date: May 26, 2022

Related U.S. Application Data
(60) Provisional application No. 62/995,159, filed on Jan. 16, 2020.

(51) Int. Cl. A45D 34/04; A45D 40/26

(52) U.S. Cl. CPC A45D 34/04; A45D 40/26; A45D 2034/005

(57) ABSTRACT

A fluid discharger and applicator device for uniform distribution or application of the fluid for treating the skin or any surface is disclosed. The fluid discharger and applicator device comprises a handle and an applicator comprising a flexible application head able to absorb fluid and an application head holder for holding the application head. The handle of the fluid discharger and applicator device is defined with a cavity to store a fluid, and the application fluid is able to enter the application head so as to be applied (or application) on skin of a user.

### BACKGROUND OF THE INVENTION

### A. Technical Field

The present invention generally relates to a hand-held fluid discharger and applicator device configured for discharging and applying a fluid, and more specifically relates to a device for discharging and applying a fluid to the skin, or in any surface for household use and so on. The present invention also relates to a method of manufacturing a fluid discharger and applicator device, a method of assembling a fluid discharger and applicator device, and a system for assembling a fluid discharger and applicator device.

### B. Description of Related Art

Generally, as part of a wet shaving process or a skin caring and cosmetic process, a user applies a skin preparation or a fluid to their skin using a brush, or their hands prior to shaving their hair in the skin, or for treating their skin, or for treating any surfaces. The preparation or fluids may be soap, foam, gel, lotion, etc. For hygienic treatment, it is recommended to apply the fluid on the skin using a sponge rather than using their bare fingers. Further, holding the sponges using the bare-fingers for treatment is also unhygienic, as it will restrict the free airflow through the sponge. This action further obstructs effective penetration of fluid through the skin pores. In addition, these sponges get wet and become too hard to hold it through the bare fingers during the application over the skin.

The use of cosmetic wedges in bare hand to enable a person to dip and apply the fluid for treating their skin, or any surfaces is well-known in the art. As further known in the art, the shaving system comprises skin care facilities by using soft sponge like material or a lubricating strip coupled to the shaving cartridge for easy dip and application of shaving lotion, creams, moisturizer and other cosmetic thereof. However, this is a messy and lengthy process, which requires the use of multiple products/tools such as cosmetic wedges bare hand or shaving cartridge to enable a person to dip and apply the fluid for treating their skin, or any surfaces. Even though the present art eliminates the use of sponges, brushes, lubricating strip or the like during shaving process, but they lack in uniform discharge and application of the fluids to the user's skin or on any surfaces.

Therefore, there is a need for a device capable of uniformly discharging, distributing or applying the fluid for treating the skin or any other surfaces, and convenient for using and carrying.

### SUMMARY OF THE INVENTION

The present invention relates to a fluid discharger and applicator device. In one embodiment, the device comprises a handle and an applicator connected to one end of the handle. The applicator comprises a flexible application head which is able to absorb fluid and an application head holder for holding the application head.

In one embodiment, the handle defines a cavity therein. The cavity is used for storing application fluid. The application fluid from the cavity is able to enter the application head.

In one embodiment, the application fluid is stored in an application fluid core or stored in an application fluid cassette. The application fluid core or body of the cassette is flexible and able to absorb the fluid and arranged in the cavity. A pressing part is provided on the handle for squeezing the application fluid core or the cassette.

In one embodiment, a knob is provided on the pressing part.

In one embodiment, the cavity is in fluid communication with the application head by a channel.

In one embodiment, a check valve is provided in the channel.

In one embodiment, the application fluid is filled in a cassette, a body of which is flexible and able to be loaded in the cavity of the handle, the handle is provided with a pressing part for squeezing the cassette.

In one embodiment, a knob is provided on the pressing part.

In one embodiment, the cavity is communicated with the application head through a pipeline which is able to puncture the cassette.

In one embodiment, the portion of the handle corresponding to the cavity is made of flexible material.

In one embodiment, the handle and the applicator are of integrated structure.

In one embodiment, the application head holder is a shell including an open end, with the application head partially arranged in the shell to expose an application side there from. The end of the shell opposite to the open end is provided with an orifice for fluid communication with the cavity of the handle, the open end of the shell is releasably sealed by an airtight cover.

In one embodiment, at least one of the handle and the shell is manufactured with one or more of plastic, eco-friendly material, paper-type material, saw dust type material, acrylic, even glass, bio-degradable materials, recyclable material.

In one embodiment, a pressing part is provided on the portion of the application head holder opposite to the application side, which is able to press out the fluid in the application head, the pressing part provided on the portion of the application head holder is formed of flexible material.

In one embodiment, the handle is detachably connected with the applicator.

In one embodiment, the handle is elastically and fixedly connected with the applicator, so that the fluid discharger and applicator device can be slightly bent during use.

In one embodiment, the application side is at an angle from 45 degrees to 135 degrees with a surface to be applied.

The present invention further relates to a method of manufacturing a fluid discharger and applicator device. The method includes following steps: processing molds by molding, producing various components of the fluid discharger and applicator device using molds, producing application head and application fluid core or application fluid cassette, and assembling components with the application heads and the application fluid cores or the application fluid cassettes to form the fluid discharger and applicator device.

In one embodiment, the step of producing application heads and application fluid cores includes producing uniform specifications of the application heads and application fluid cores by cutting a flexible material which can absorb fluid.

In one embodiment, the application fluid cassettes are produced as uniform specifications of fluid pouches.

In one embodiment, the method further includes a step of injecting application fluid into the application head and application fluid core, or enclosing application fluid into the application fluid cassettes.

In one embodiment, the various components of the fluid discharger and applicator device include an applicator and a handle; and the applicator and the handle are produced separately by molds.

The present invention further relates to a method of assembling a fluid discharger and applicator device, wherein the method comprises the steps of:
a) Conveying a first shell,
b) Installing a bottom sealing cover on the application side of the first shell;
c) Installing an application head into the first shell and onto the sealing cover;
d) Installing a top sealing cover onto the application head;
e) Install a second shell onto the first shell to form the fluid discharger and applicator device.

In one embodiment, the method further includes steps between a) and b):
f) installing a sealing plate onto the bottom of a cavity in the first shell, and
g) installing application fluid core or application fluid cassette on the sealing plate.

In one embodiment, the method, when the application fluid cassette was installed on the sealing plate, between g) and b), further includes:
h) installing a pressing part on the application fluid core or application fluid cassette.

In one embodiment, the method, between h) and b) ,further includes : i) installing a check valve into the channel.

In one embodiment, the method further includes: j) installing a knob on the pressing part.

In one embodiment, the second shell is installed onto the first shell in a detachable way or in none detachable way.

In one embodiment, wherein the first shell and the second shell form a handle.

The present invention further relates to a system for assembling a fluid discharger and applicator device. The system comprise a first flow line, for conveying a first shell; a second flow line, for conveying a second shell; a third flow line, for conveying a bottom sealing cover; a fourth flow line, for conveying an application head; and a fifth flow line, for conveying a top sealing cover. The first flow line and the third flow line meet in a first station, for installing the bottom sealing cover on the application side of the first shell. The first flow line and the fourth flow line meet in a second station following the first station, for installing the application head into the first shell and onto the sealing cover. The first flow line and the fifth flow line meet in the third station following the second station, for installing the top sealing cover onto the application head. The first flow line meet the second flow line in the fourth station following the third station, for install the second shell onto the first shell to form the fluid discharger and applicator device.

In one embodiment, the system further comprises: a sixth flow line for conveying a sealing plate; a seventh flow line for conveying an application fluid core or an application fluid cassette. The first flow line and the sixth flow line meet in the fifth station preceding the first station, for installing the sealing plate onto the bottom of a cavity in the first shell. The first flow line and the seventh flow line meet in the sixth station following the fifth station preceding the first station, for installing application fluid core or application fluid cassette on the sealing plate.

In one embodiment, when the application fluid cassette was installed on the sealing plate, the system further comprises an eighth flow line for conveying a pressing part. The first flow line and the eighth flow line meet in a seventh station following the sixth station preceding the first station, for installing the pressing part on the application fluid core or application fluid cassette.

In one embodiment, the system further comprises a ninth flow line for conveying a check vale. The first flow line and the ninth flow line meet in an eighth station following the seventh station preceding the first station, for installing the check valve into the channel.

In one embodiment, the system further comprises a tenth flow line for conveying a knob. The first flow line and the tenth flow line meet in a ninth station following the eighth station preceding the first station, for installing the knob on the pressing part.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1A-1C show schematic view of one embodiment of a fluid discharger and applicator device according to the present invention.

FIG. 2A-2C show schematic view of another one embodiment of a fluid discharger and applicator device according to the present invention.

FIG. 3A-3D show schematic view of another one embodiment of a fluid discharger and applicator device according to the present invention

FIG. 4 shows a flow diagram of a method of manufacturing a fluid discharger and applicator device according to the present invention

FIG. 5 shows a flow diagram of a method of assembling a fluid discharger and applicator device according to the present invention.

FIG. 6 shows schematic view of one system for assembling a fluid discharger and applicator device.

FIG. 7 shows schematic view of another one system for assembling a fluid discharger and applicator device.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that evolve within the meaning and range of equivalency of the claims are to be embraced within their scope.

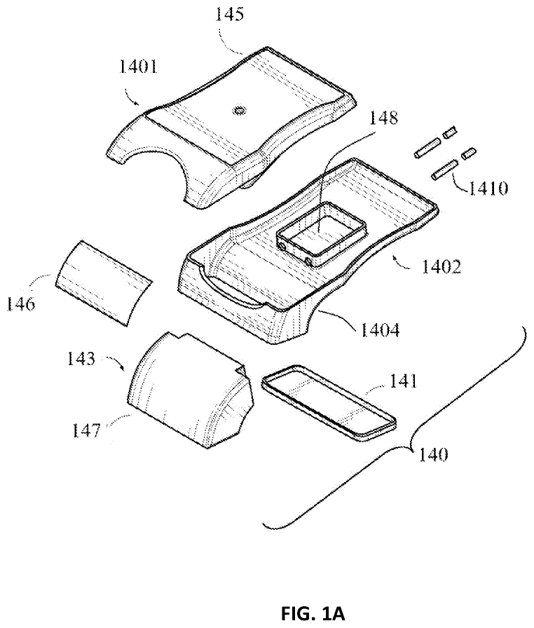

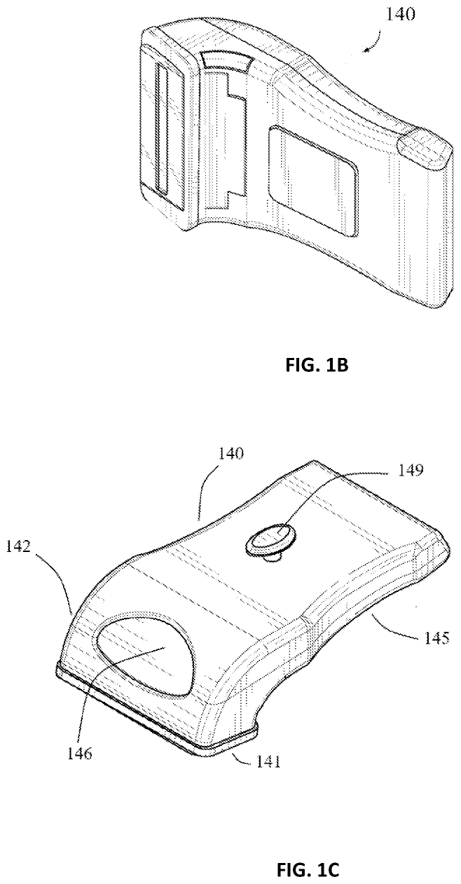

FIG. 1A shows an exploded view of an embodiment of a fluid discharger and applicator device 140, which more clearly shows the structure of the fluid discharger and applicator device 140. FIGS. 1B and 1C show perspective views of the fluid discharger and applicator device of FIG. 1A. The device 140 mainly comprises a first component 1401 and a second component 1402. When assembled together, the first component 1401 and the second component 1402 can form a frame of the device. The fluid discharger and applicator device according to this embodiment may be a multi-use disposable type. For example, an user may bring this device for use on the weekend.

In this embodiment, the device140 comprises a handle 145 and an applicator 142 connected to one end of the handle 145. The applicator 142 comprises a flexible application head 143 which is able to absorb fluid and an application head holder 1404 for holding the application head 143. The application head 143 may be a sponge. Alternatively, the application head 143 may be made up of materials like rubber, as long as it can absorb the application fluid and be pressed out the fluid. The application head 143 comprises an application side147. The handle 145 is defined a cavity 148 therein.

The application fluid may be any viscous material, including liquid or semi-solid, which has a flowing viscosity. For example, the application fluid may be oil, lotion, cream, serum. In this embodiment, the cavity 148 may be used for storing the application fluid. Optionally, the application head also may be prefilled with fluid to be applied. The cavity may be in fluid communication with the application head by a channel or a duct. In the embodiment as shown in FIGS. 1A-1C, the cavity is in fluid communication with the application head by two (channels or) ducts 1410. The application head holder may be a shell with an open end. The open end is releasably sealed by an airtight cover 141, in order to prevent the application head from drying out.

As shown in FIG. 1A, a pressing part 146 is provided on the application head 143, so as to press out the application fluid, as needed.

During using the device, the user may depress the pressing part 146 to press out an amount of the fluid to be applied onto a surface, for example, the skin of the user. As the device is used, the application fluid in the cavity may flow to the application head through the duct and thus is absorbed by the application head, and then applied on the skin of a user, as needed.

A knob 149 is provided on the handle 145, which is used to pressing an application fluid core (not shown) in the cavity.

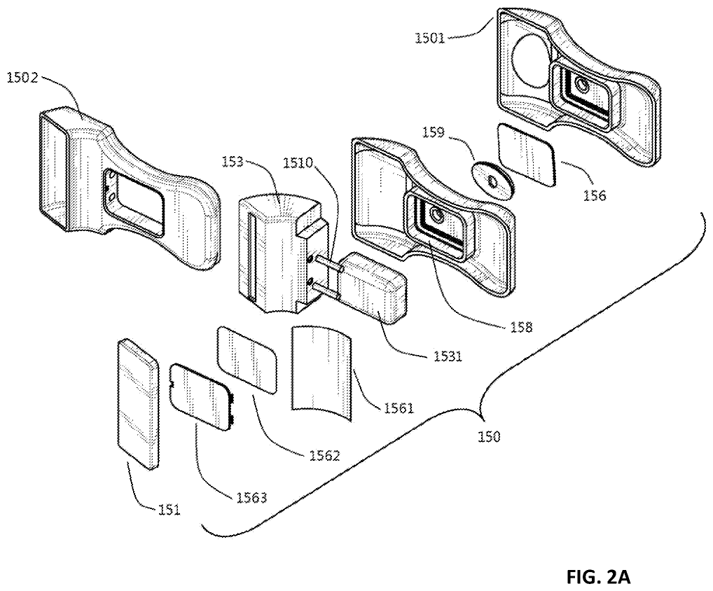

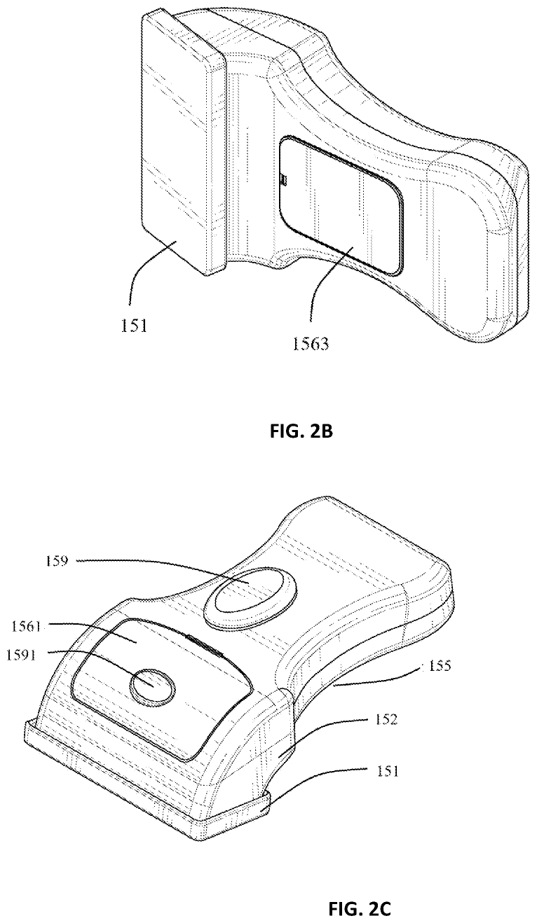

FIGS. 2A-2C show one embodiment of a fluid discharger and applicator device 150. Specially, as shown in FIG. 2A, the device 150 comprises a first component 1501 and a second component 1502. When assembled together, the first component 1501 and the second component 1502 can form a frame of the device.

In particular, the first component 1501 may be assembled with the second component 1502, so as to form the handle 155 and the application head holder152. The handle includes a cavity 158. The application head holder 152 includes a notch in the first component and an open end in the second component. The open end is releasably sealed by an airtight cover 151, in order to prevent the application head 153 from drying out. A pressing part 1561 may be provided on the application head 153 to press out the application fluid. In one embodiment, a knob 1591 is provided on the pressing part 1561 to facilitate the user to press the application head 153. A sealer 1562 is provided on the bottom plate 1563 of the cavity 158. A pressing part 156 is provided in the handle for squeezing the application fluid core 1531. In one embodiment, a knob 159 is provided on the pressing part 156 to facilitate the user to press the application fluid core 1531.

In this embodiment, the application fluid stored in the cavity 158 may be stored in an application fluid core 1531. The application fluid core1531 is arranged in the cavity. The application fluid core 1531 may be a flexible body, which may be formed from any material that is able to absorb the application fluid. The application fluid core 1531 may be produced from the same material as that of the application head 153. Of course, the application fluid core 1531 may be produced from different material from that of the application head153. The application fluid from the cavity 158 is able to enter the application head 153 through a channel or a duct. In this embodiment, the application fluid from the cavity enters the application head through two ducts 1510. The knob 159 may be disposed outside of the handle 155 such that the user may depress the knob 159 and thus the pressing part to press out the application fluid for entering the application head 153 through the ducts 1510.

In one embodiment, the cavity is in fluid communication with the application head 153 by a channel (not shown). In one embodiment, a check valve may be provided in the channel to prevent the application fluid from flowing back to the application core 1531.

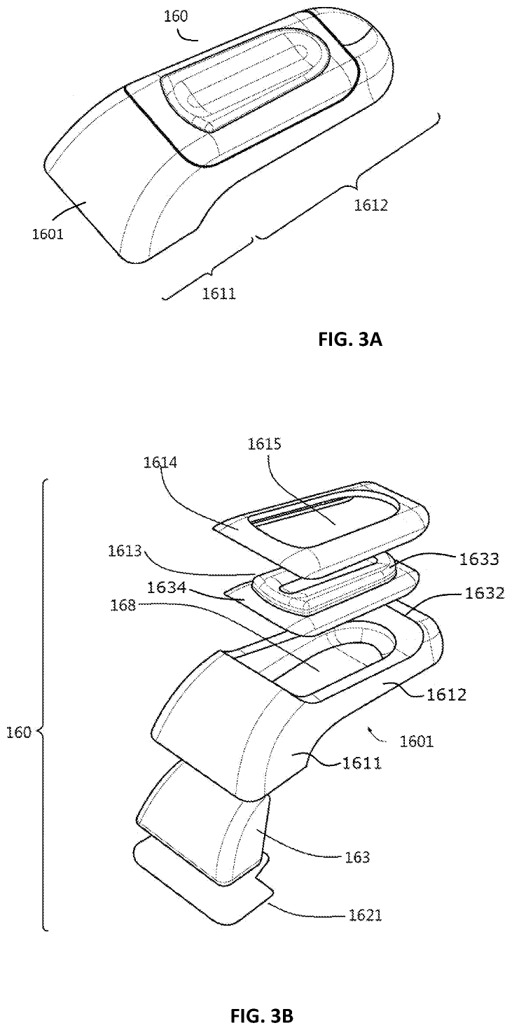

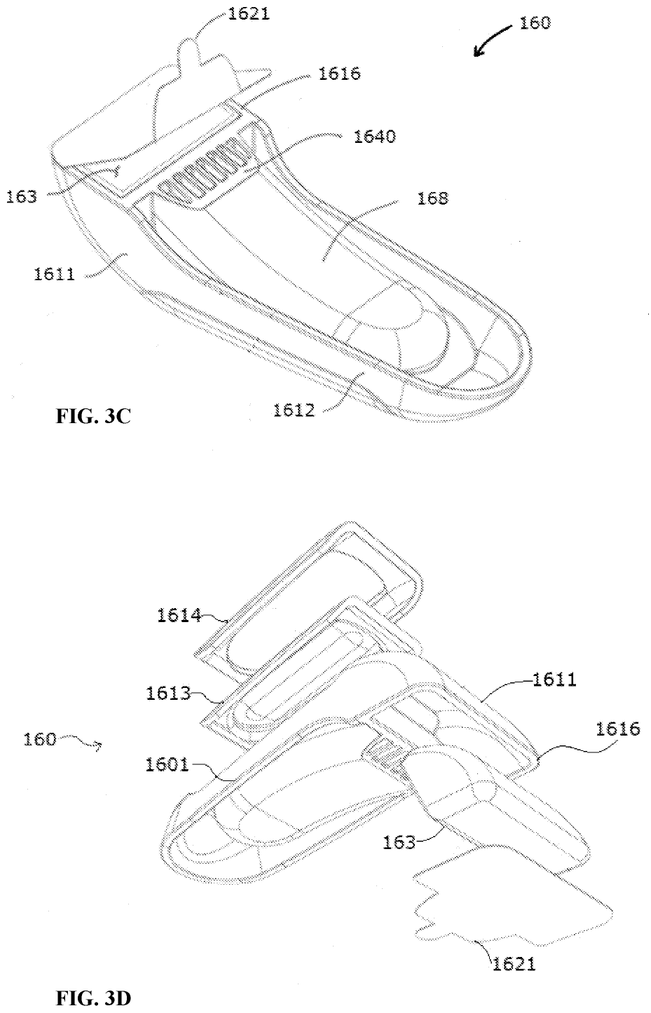

FIGS. 3A-3D shows one embodiment of a fluid discharger and applicator device 160. Specially, as shown in FIG. 3B, the device 160 comprises a casing 1601. The casing 1601 comprises a handle 1612 and an applicator 1611. That is, the handle and the applicator are of integrated structure. The applicator 1611 unitary with the handle 1612 and terminating in an open applicator face 1616. A grated opening 1640 is disposed adjacent an open applicator face 1616 so as to enable the user to dispense the personal care product from the grated opening 1640, and then to move about the device 160 so that the application head 163 spreads the personal care product as desired. The applicator 1611 is configured so as to enable a user to conveniently place the open applicator face 1616 against the desired body region without requiring the user to hold the device 160 at an awkward angle.

As shown in FIG. 3B, a cavity 168 is formed in the handle 1612. The cavity 168 may be used for loading the application fluid cassette 1613. The application fluid cassette 1613 may be prefilled with an application fluid to be applied. The handle 1611 may be provided with a pressing part for squeezing the cassette 1613. Alternatively, in this embodiment, a cover 1614 is provided for cover the cavity 168, which has a notch 1615. The device 160 further includes the cover 1614 configured to be flush fit onto a casing recess 1632. The top surface of the cassette 1613 is exposed to facilitate the user pressing the cassette 1613 to transfer the fluid into the application head 163. Accordingly, the cassette 1613 may be made of a flexible material at least in its top surface. In one embodiment, the cavity 168 is in fluid communicated with the application head 163 through a pipeline (not shown) which is able to puncture the cassette 1613.

In one embodiment, the handle corresponding to the cavity 168 may be made of flexible material. The user may depress the portion for squeezing the cassette 1613.

In one embodiment, the cavity 168 is in fluid communicated with the application head 163 through a pipeline (not shown) which is able to puncture the cassette 1613 exposed to facilitate the user pressing the cassette 1613 to transfer the fluid into the application head 163.

In one embodiment, the application head holder is a shell including an open end, with an application head 163 partially arranged in the shell to appropriately expose an application side there from. The end of the shell opposite to the open end is provided with an orifice for fluid communication with the cavity of the handle. The open end of the shell is releasably sealed by an airtight cover 1621.

Referring to FIG. 3B, it can be seen that fluid cassette 1613 is generally hat-shaped, with a resilient mesa 1633 extending above a perimeter tab 1634. The resilient mesa 1633 is thus configured to protrude through the notch 1615 enclosed by the cover 1614. In the example provided, the resilient mesa 1633 and the notch 1615 are substantially rectangular in shape, although it should be understood that different geometric shapes can be used including, for example, circular, oval, or triangular.

In one embodiment, at least one of the handle and the shell is manufactured with one or more of plastic, eco-friendly material, paper-type material, saw dust type material, acrylic, even glass, bio-degradable materials, recyclable material.

In one embodiment, a pressing part is provided on the portion of the application head holder opposite to the application side, which is able to press out the fluid in the application head. The pressing part provided on the portion of the application head holder is formed of flexible material.

In one embodiment, the connection portion between the handle portion and the application portion is elastic and fixable, so that the fluid discharger and applicator device can be slightly bent during use. In one embodiment, the pressing part provided on the portion of the application head holder is formed of flexible material. This attribute will make the user feel comfortable.

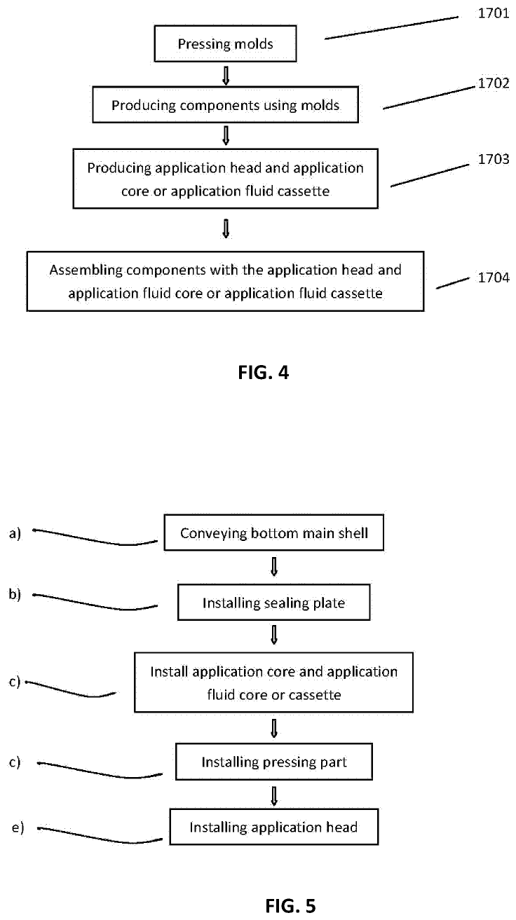

FIG. 4 shows a method of manufacturing a fluid discharger and applicator device according to the present invention.

At step 1701, the molds are processed by molding. The type and shape of the mold depend on those of component consisting of the fluid discharger and applicator device as needed

At step 1702, various components of the fluid discharger and applicator device are produced using respective mold. In one embodiment, the various components of the fluid discharger and applicator device may include an applicator, a handle, a pressing part, an airtight cover and other accessory parts, for example. The handle portion and the applicator portion may be separate parts or a single part, depending on the type of the fluid discharger and applicator device.

At step 1703, the application head and application fluid core or application fluid cassette are produced. In one embodiment, the step of producing application heads and application fluid cores includes producing uniform specifications of the application heads and application fluid cores by cutting a flexible material which can absorb fluid. In one embodiment, the application fluid cassettes are produced as uniform specifications of fluid pouches.

At step 1704, the components as produced in step 1702 are assembled with the application heads and the application fluid cores or the application fluid cassettes as produced in step 1703 to form a finished fluid discharger and applicator device.

In one embodiment, the method further includes a step of injecting application fluid into the application head and application fluid core, or enclosing application fluid into the application fluid cassettes.

The present invention further relates to a method of assembling a fluid discharger and applicator device, as shown in FIG. 5. The method comprises the steps of:
a) Conveying first shell,
b) Installing bottom sealing cover on the application side of the first shell;
c) installing an application head into the first shell and onto the sealing cover;
d) Installing top sealing cover onto the application head;
e) Install a second shell onto the first shell to form the fluid discharger and applicator device.

In one embodiment, the method further includes steps between a) and b):
f) Installing sealing plate onto the bottom of a cavity in the first shell, and
g) Installing application fluid core or application fluid cassette on the sealing plate.

In one embodiment, the method, when the application fluid cassette was installed on the sealing plate, between g) and b), further includes:
h) Installing a pressing part on the application fluid core or application fluid cassette.

In one embodiment, the method, between h) and b), further includes: i) installing a check valve into the channel.

In one embodiment, the method further includes: j) installing a knob on the pressing part.

In one embodiment, the second shell is installed onto the first shell in a detachable way or in none detachable way.

In one embodiment, the first shell and the second shell form a handle.

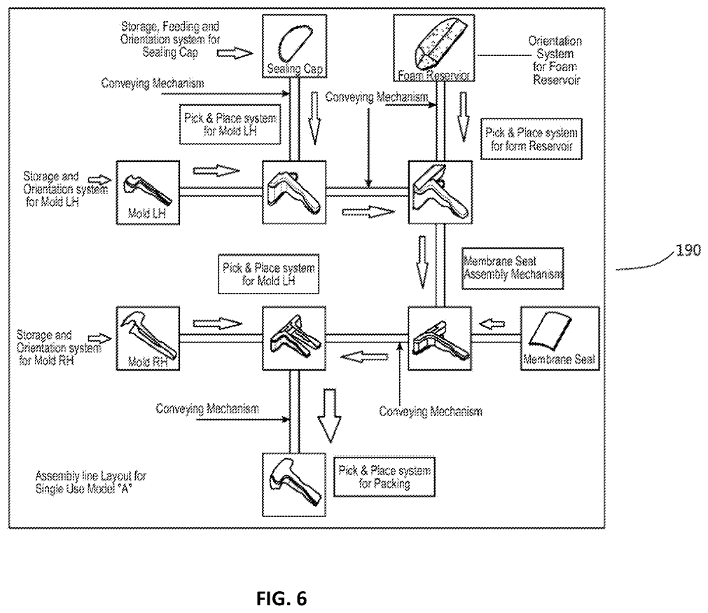

The present invention further relates to a system for assembling a fluid discharger and applicator device, as shown in Fig.6. The system190 comprise a first flow line, for conveying a first shell; a second flow line, for conveying a second shell; a third flow line, for conveying a bottom sealing cover; a fourth flow line, for conveying an application head; and a fifth flow line, for conveying a top sealing cover. The first flow line and the third flow line meet in a first station, for installing the bottom sealing cover on the application side of the first shell. The first flow line and the fourth flow line meet in a second station following the first station, for installing the application head into the first shell and onto the sealing cover. The first flow line and the fifth flow line meet in the third station following the second station, for installing the top sealing cover onto the application head. The first flow line meets the second flow line in the fourth station following the third station, for install the second shell onto the first shell to form the fluid discharger and applicator device.

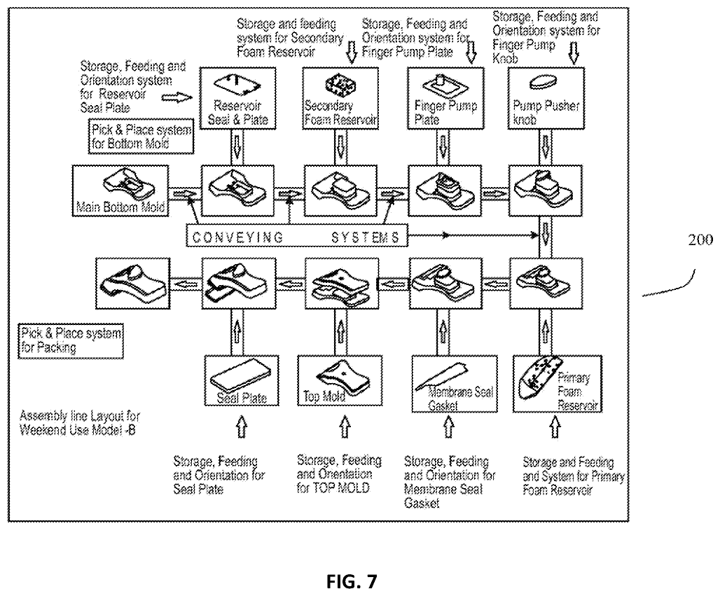

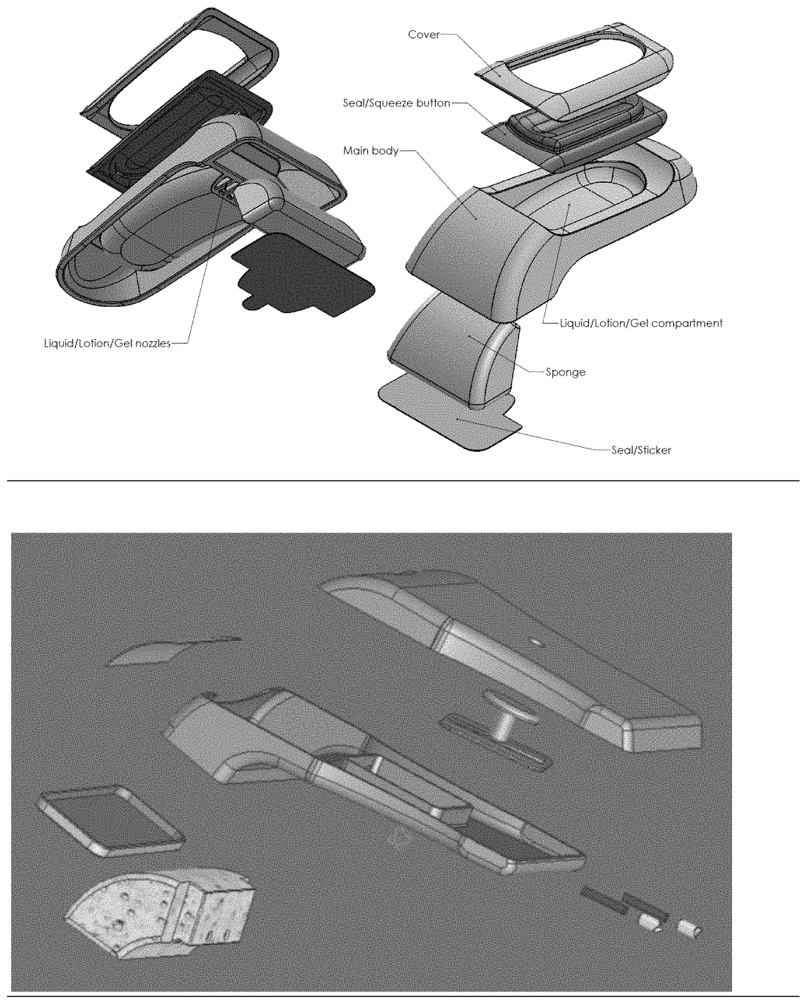

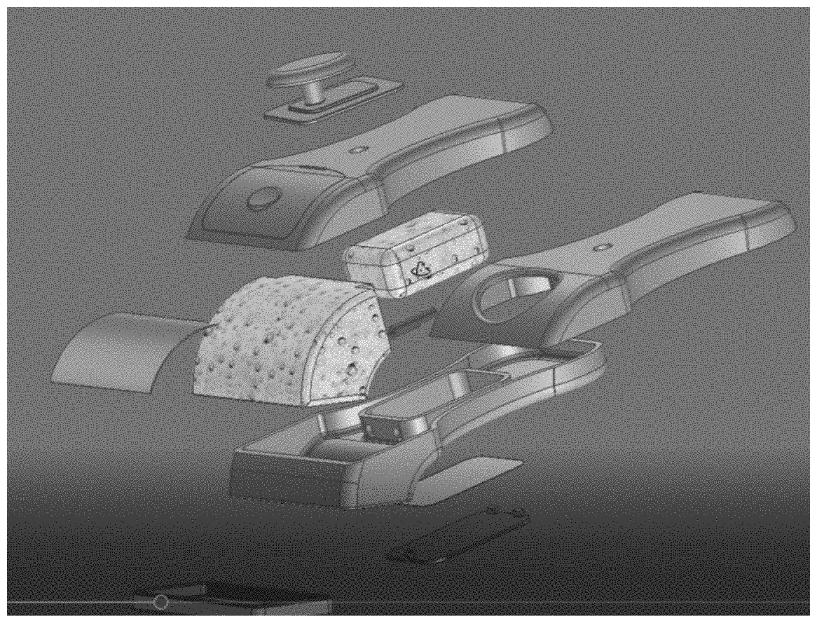

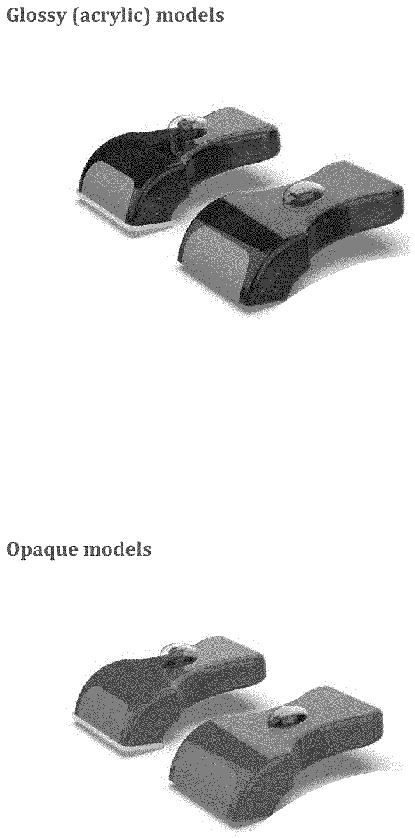

FIG. 7 shows schematic view of another one system for assembling a fluid discharger and applicator device. The system 200, in addition to the five flow lines described according to FIG. 6, the system 200 further comprises: a sixth flow line for conveying a sealing plate; a seventh flow line for conveying an application fluid core or an application fluid cassette. The first flow line and the sixth flow line meet in the fifth station preceding the first station, for installing the sealing plate onto the bottom of a cavity in the first shell. The first flow line and the seventh flow line meet in the sixth station following the fifth station preceding the first station, for installing application fluid core or application fluid cassette on the sealing plate.

When the application fluid cassette was installed on the sealing plate, the system 200 further comprises an eighth flow line for conveying a pressing part. The first flow line and the eighth flow line meet in a seventh station following the sixth station preceding the first station, for installing the pressing part on the application fluid core or application fluid cassette.

The system 200 further comprises a ninth flow line for conveying a check vale. The first flow line and the ninth flow line meet in an eighth station following the seventh station preceding the first station, for installing the check valve into the channel.

The system 200 further comprises tenth flow line for conveying a knob. The first flow line and the tenth flow line meet in a ninth station following the eighth station preceding the first station, for installing the knob on the pressing part.

The foregoing descriptions comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Although specific terms may be employed herein, they are used only in generic and descriptive sense and not for purposes of limitation. Accordingly, the present invention is not limited to the specific embodiments illustrated herein.

What is claimed is:

1. A fluid discharger and applicator device, comprising:
a casing with a handle, and an applicator connected to one end of the handle,
the applicator comprising:
a flexible application head which is able to absorb fluid, and
an application head holder for holding the application head,
wherein the handle defines a cavity therein, the cavity is used for storing application fluid, and
the application fluid is able to enter the application head,
wherein the application fluid is absorbed in an application fluid core, which is flexible and able to be arranged in the cavity, a pressing part is provided on the handle for squeezing the application fluid core.

2. The fluid discharger and applicator device according to claim 1, wherein a knob is provided on the pressing part.

3. The fluid discharger and applicator device according to claim 1, wherein the cavity is in fluid communication with the application head by a channel.

4. The fluid discharger and applicator device according to claim 3, wherein a check valve is provided in the channel.

5. A fluid discharger and applicator device, comprising:
a casing with a handle, and an applicator connected to one end of the handle,
the applicator comprising:
a flexible application head which is able to absorb fluid, and
an application head holder for holding the application head,
wherein the handle defines a cavity therein, the cavity is used for storing application fluid, and the application fluid is able to enter the application head;
wherein a pressing part is provided on the portion of the application head holder opposite to an application side, which is able to press out the fluid in the application head, the pressing part provided on the portion of the application head holder is formed of flexible material.

6. The fluid discharger and applicator device according to claim 5, wherein the application fluid is absorbed in an application fluid core, which is flexible and able to be arranged in the cavity, a pressing part is provided on the handle for squeezing the application fluid core.

7. The fluid discharger and applicator device according to claim 6, wherein a knob is provided on the pressing part.

8. The fluid discharger and applicator device according to claim 6, wherein the cavity is in fluid communication with the application head by a channel.

9. The fluid discharger and applicator device according to claim 8, wherein a check valve is provided in the channel.

10. The fluid discharger and applicator device according to claim 5, wherein the application fluid is filled in a cassette, a body of which is flexible and able to be loaded in the cavity of the handle, the handle is provided with a pressing part for squeezing the cassette.

11. The fluid discharger and applicator device according to claim 10, wherein a knob is provided on the pressing part.

12. The fluid discharger and applicator device according to claim 10, wherein the cavity is communicated with the application head through a pipeline which is able to puncture the cassette.

13. The fluid discharger and applicator device according to claim 5, wherein the portion of the handle corresponding to the cavity is made of flexible material.

14. The fluid discharger and applicator device according to claim 5, wherein the handle and the applicator are of integrated structure.

15. The fluid discharger and applicator device according to claim 5, wherein the application head holder is a shell including an open end, with the application head partially arranged in the shell to expose an application side there from, and the end of the shell opposite to the open end is provided with an orifice for fluid communication with the cavity of the handle, the open end of the shell is releasably sealed by an airtight cover.

16. The fluid discharger and applicator device according to claim 15, wherein at least one of the handle and the shell is manufactured with one or more of plastic, eco-friendly material, paper-type material, saw dust type material, acrylic, even glass, bio-degradable materials, recyclable material.

17. The fluid discharger and applicator device according to claim 5, wherein the handle is detachably connected with the applicator.

18. The fluid discharger and applicator device according to claim 5, wherein the handle is elastically and fixedly connected with the applicator, so that the fluid discharger and applicator device can be slightly bent during use.

19. The fluid discharger and applicator device according to claim 5, wherein an application side is at an angle from 45 degrees to 135 degrees with an applicator surface to be applied.

20. A fluid discharger and applicator device, comprising:
a casing with a handle, and an applicator connected to one end of the handle,
the handle comprising:
a cover enclosing a notch,
a casing recess shaped so as to allow flush placement,
a fluid cassette disposed between the cover and the casing recess,
a resilient mesa in the fluid cassette configured to protrude through the notch, and
a cavity formed in the handle for storing an application fluid and loading application fluid in the fluid cassette.

21. The fluid discharger and applicator device according to claim 20, wherein the fluid cassette including a resilient mesa and a perimeter tab.

22. The fluid discharger and applicator device according to claim 20, wherein the resilient mesa sized and shaped so as to protrude through the notch so a user is enabled to dispense the fluid through a grated opening by applying pressure to the resilient mesa.

23. The fluid discharger and applicator device according to claim 22, wherein the grated opening is substantially coplanar with an open applicator face and disposed of adjacent to the open applicator face.

24. The fluid discharger and applicator device according to claim 20, wherein the cavity is in fluid communicating with the application head through a channel between the cavity and the fluid cassette.

25. The fluid discharger and applicator device according to claim 20, wherein the fluid cassette is flexible, and wherein a pressing part is provided on the handle for squeezing the fluid cassette.

26. The fluid discharger and applicator device according to claim 20, wherein the applicator is unitary with the handle.

27. The fluid discharger and applicator device according to claim 20, wherein at least one of the handle and the applicator is manufactured with one or more of plastic, eco-friendly material, paper-type material, saw dust type material, acrylic, even glass, bio-degradable materials, recyclable material.

28. A method of manufacturing a fluid discharger and applicator device, including:
Processing molds by molding,
Producing various components of the fluid discharger and applicator device using molds,
Producing application head and application fluid core or application fluid cassette, and
Assembling components with the application heads and the application fluid cores or the application fluid cassettes to form the fluid discharger and applicator device,
wherein the various components of the fluid discharger and applicator device includes an application head holder and a handle, a pressing part is provided on the portion of the application head holder opposite to an application side, which is able to press out fluid in the application head, and the pressing part provided on the portion of the application head holder is formed of flexible material.

29. The method according to claim 28, wherein producing application heads and application fluid cores includes producing uniform specifications of the application heads and application fluid cores by cutting a flexible material which can absorb fluid.

30. The method according to claim 28, wherein the application fluid cassettes are uniform specifications of fluid pouches.

31. The method according to claim 28, further includes injecting application fluid into the application head and application fluid core, or enclosing application fluid into the application fluid cassettes.

32. The method according to claim 28, wherein the application head holder and the handle are produced separately by molds.

33. A method of assembling a fluid discharger and applicator device, wherein the method comprises the steps of:
a) conveying a first shell,
b) Installing a bottom sealing cover on an application side of the first shell;
c) installing an application head into the first shell and onto the bottom sealing cover;
d) Installing a top sealing cover onto the application head;
e) Install a second shell onto the first shell to form the fluid discharger and applicator device,
wherein a pressing part is provided on the portion of the second shell, which is able to press out fluid in the application head, and the pressing part provided on the portion of the second shell is formed of flexible material.

34. The method according to claim 33, between a) and b), further includes:
f) Installing a sealing plate onto the bottom of a cavity in the first shell, and
g) Installing application fluid core or application fluid cassette on the sealing plate.

35. The method according to claim 34, when the application fluid cassette was installed on the sealing plate, between g) and b), further includes:
h) Installing a pressing part on the application fluid core or application fluid cassette.

36. The method according to claim 35, between h) and b) further includes:
i) Installing a check valve into the channel.

37. The method according to claim 33, further includes:
j) Installing a knob on the pressing part.

38. The method according to claim 33, wherein the second shell is installed onto the first shell in a detachable way or in none detachable way.

39. The method according to claim 33, wherein the first shell and the second shell form a handle.

40. A system for assembling a fluid discharger and applicator device, comprising:
a first flow line, for conveying a first shell;
a second flow line, for conveying a second shell;
a third flow line, for conveying a bottom sealing cover;
a fourth flow line, for conveying an application head; and
a fifth flow line, for conveying a top sealing cover;
wherein, the first flow line and the third flow line meet in a first station, for installing the bottom sealing cover on the application side of the first shell; the first flow line and the fourth flow line meet in a second station following the first station, for installing the application head into the first shell and onto the sealing cover; the first flow line and the fifth flow line meet in the third station following the second station, for installing the top sealing cover onto the application head; and the first flow line meet the second flow line in the fourth station following the third station, for install the second shell onto the first shell to form the fluid discharger and applicator device, and
wherein a pressing part is provided on the portion of the second shell, which is able to press out fluid in the application head, and the pressing part provided on the portion of the second shell is formed of flexible material.

41. The system of according to claim 40, wherein the system further comprises:
a sixth flow line for conveying a sealing plate;
a seventh flow line for conveying an application fluid core or an application fluid cassette;
wherein the first flow line and the sixth flow line meet in the fifth station preceding the first station, for installing the sealing plate onto the bottom of a cavity in the first shell;
and the first flow line and the seventh flow line meet in the sixth station following the fifth station preceding the first station, for installing application fluid core or application fluid cassette on the sealing plate.

42. The system of according to claim 41, wherein when the application fluid cassette was installed on the sealing plate, the system further comprises:
an eighth flow line for conveying a pressing part,
wherein the first flow line and the eighth flow line meet in a seventh station following the sixth station preceding the first station, for installing the pressing part on the application fluid core or application fluid cassette.

43. The system of according to claim 42, wherein the system further comprises:
a ninth flow line for conveying a check vale, wherein the first flow line and the ninth flow line meet in an eighth station following the seventh station preceding the first station, for installing the check valve into the channel.

44. The system of according to claim 43, wherein the system further comprises:
a tenth flow line for conveying a knob, wherein the first flow line and the tenth flow line meet in a ninth station following the eighth station preceding the first station, for installing the knob on the pressing part.
